(19) United States
(12) Patent Application Publication
(10) Pub. No.: US 2025/0349418 A1
(43) Pub. Date: Nov. 13, 2025

(54) ABNORMALITY PROCESSING SYSTEM AND ABNORMALITY PROCESSING METHOD

(71) Applicants: KABUSHIKI KAISHA TOSHIBA, Minato-ku, Tokyo (JP); TOSHIBA ENERGY SYSTEMS & SOLUTIONS CORPORATION, Kawasaki-shi, Kanagawa (JP)

(72) Inventors: Keiko OKAYA, Tokyo (JP); Hiromasa ITOH, Gyoda (JP)

(73) Assignees: KABUSHIKI KAISHA TOSHIBA, Minato-ku, Tokyo (JP); TOSHIBA ENERGY SYSTEMS & SOLUTIONS CORPORATION, Kawasaki-shi, Kanagawa (JP)

(21) Appl. No.: 19/276,124

(22) Filed: Jul. 22, 2025

Related U.S. Application Data
(63) Continuation of application No. PCT/JP2023/045597, filed on Dec. 20, 2023.

(30) Foreign Application Priority Data
May 15, 2023 (JP) 2023-079822

(51) Int. Cl. G16H 40/63

(52) U.S. Cl. CPC G16H 40/63

(57) ABSTRACT

According to the present embodiment, an abnormality processing system includes one or more management computers connected to a plurality of computers configured to process information relating to a particle beam treatment apparatus through a network, wherein the management computer is configured to; acquire processing information indicating a current state of the particle beam treatment apparatus from a plurality of computers; detect, when at least one abnormality occurs in the particle beam treatment apparatus, an abnormality from the acquired processing information; and display, upon detecting the abnormality, at least one handling item indicating a processing method for a user to process the abnormality, which includes information enabling the user to determine whether to continue or interrupt a treatment for a patient.

### CROSS-REFERENCE TO RELATED APPLICATIONS

This application is a Continuation Application of PCT/JP2023/045597 filed on Dec. 20, 2023, and the PCT application is based upon and claims benefit of priority from Japanese Patent Application No. 2023-079822 filed on May 15, 2023, the entire contents of which are incorporated herein by reference.

### BACKGROUND OF THE INVENTION

### Field of the Invention

The present invention embodiment relates to abnormality processing technology.

### Description of the Related Art

Technology for displaying the processing method for abnormality or failure occurring in a charged particle beam apparatus has been known. Charged particle beam apparatuses have been used in various fields, in particular, particle beam treatment apparatuses have spread widely. For example, Patent Document 1 (Japanese Patent Application Publication No. 2007-324084) discloses a charged particle beam apparatus implementing an abnormality processing method.

In conventional technology, however, it takes a long time of processing when an abnormality occurs in a particle beam treatment apparatus, and therefore it is necessary for a user to determine as to whether to bring a patient out of a treatment couch or not, that is, whether to continue or interrupt treatment. This may impose a burden on the user. In addition, when interrupting treatment irrespective of rapid recovery of the particle beam treatment apparatus, it is necessary to carry out treatment on a later date, which may impose a burden on the patient.

An objective problem to be solved by the present invention is to display handling items helping user to rapidly determine as to whether to approve or disapprove continuation of treatment in case of any abnormality occurring in a particle beam treatment apparatus.

### SUMMARY OF THE INVENTION

The embodiment of the present invention refers to an abnormality processing system including at least one management computer connected to a plurality of computers for processing information relating to a particle beam treatment apparatus through a network. The management computer is configured to acquire processing information representative of the current status of the particle beam treatment apparatus from a plurality of computers; when at least one abnormality occurs in the particle beam treatment apparatus, to detect the abnormality from the processing information acquired; and upon detecting the abnormality, to display at least one handling item representative of a processing method for a user to handle the abnormality corresponding to a handling item including information causing the user to determine whether to continue or interrupt a treatment for a patient.

Another embodiment of the present invention refers to an abnormality processing method executed by at least one management computer connected to a plurality of computers for processing information relating to a particle beam treatment apparatus through a network. The management computer is configured to acquire processing information representative of the current status of the particle beam treatment apparatus from a plurality of computer; when at least one abnormality occurs in the particle beam treatment apparatus, to detect the abnormality from the processing information acquired; and upon detecting the abnormality, to display at least one handling item representative of a processing method for a user to handle the abnormality corresponding to a handling item including information causing the user to determine whether to continue or interrupt a treatment for a patient.

### BRIEF DESCRIPTION OF THE DRAWINGS

FIG. 1 is a network configuration diagram of an abnormality processing system.

FIG. 2 is a block diagram illustrating a system configuration of the abnormality processing system.

FIG. 3 is a screen image showing an exemplary display manner of handling items.

FIG. 4 is a flowchart showing an abnormality information display process.

FIG. 5 is the flowchart showing the abnormality information display process following the flowchart of FIG. 4.

FIG. 6 is a flowchart showing a log collection process.

### DETAILED DESCRIPTION OF THE INVENTION

An abnormality processing system according to the present invention embodiment includes at least one management computer connected to a plurality of computers for processing information relating to a particle beam treatment apparatus through a network. The management computer is configured to acquire processing information representative of the current status of the particle beam treatment apparatus from a plurality of computers; when at least one abnormality occurs in the particle beam treatment apparatus, to detect the abnormality from the processing information acquired; and upon detecting the abnormality, to display at least one handling item representative of a processing method for a user to handle the abnormality corresponding to a handling item including information causing the user to determine as to whether to continue or interrupt treatment for a patient.

According to the present invention embodiment, when an abnormality occurs in a particle beam treatment apparatus, it is possible to display a handling item causing a user to immediately determine as to whether to continue treatment or not.

The embodiment of an abnormality processing system and an abnormality processing method will be described below in detail with reference to the accompanying drawings.

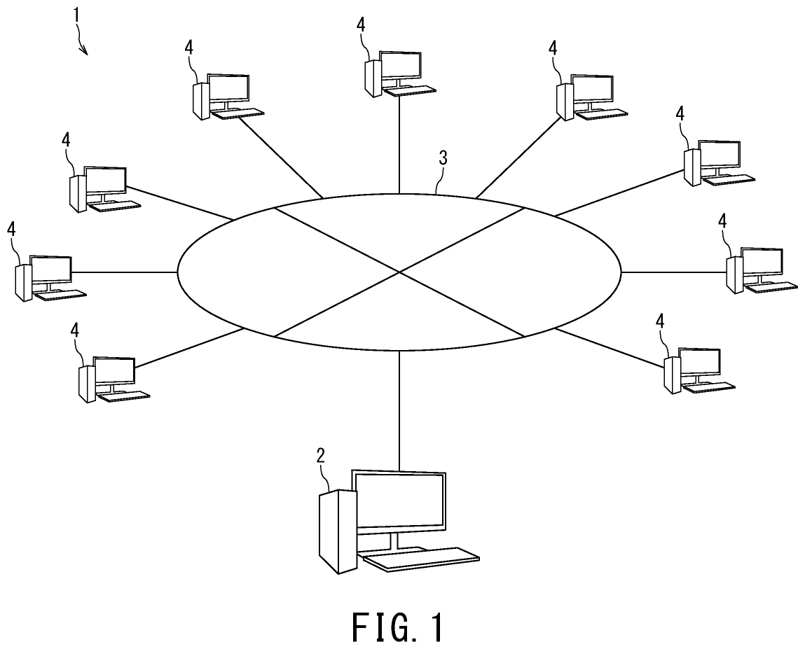

FIG. 1 shows an abnormality processing system 1 according to the present embodiment. When an abnormality occurs in a particle beam treatment apparatus (not shown), the abnormality processing system 1 provides a processing method for a user to handle the abnormality.

The abnormality processing system 1 includes a management computer 2. The management computer 2 is connected to a plurality of other computers 4 through a network 3. The other computers 4 are each configured to process information relating to a particle beam treatment apparatus. The other computers 4 are each configured to process information about the particle beam treatment apparatus, for example, information about radiation management, building facilities, accelerators, medical image processing apparatuses, and treatment room equipment/control.

The management computer 2 is configured to acquire the processing information representative of the current status of a particle beam treatment apparatus from the other computers 4, and, when an abnormality occurs in the particle beam treatment apparatus, to provide the user with handling items including information representative of a processing method.

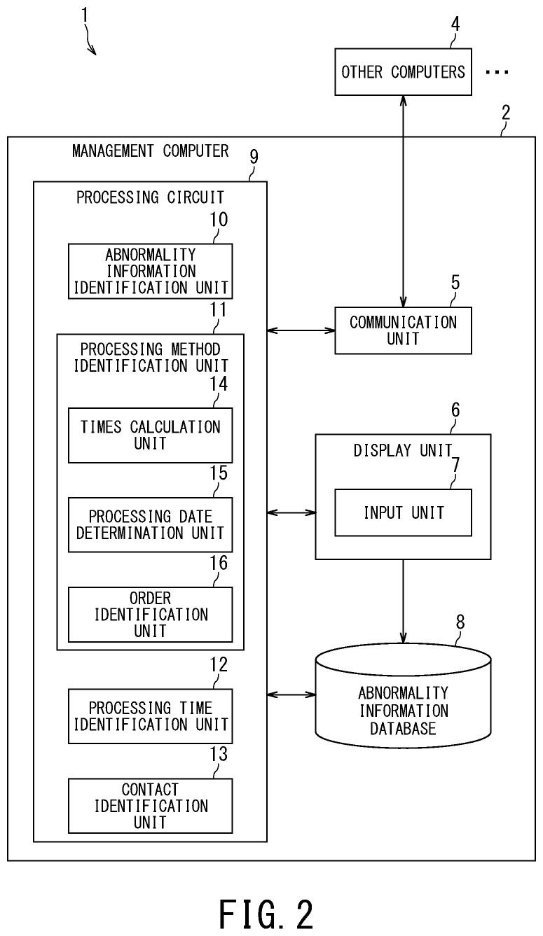

Next, the system configuration of the abnormality processing system 1 will be described with reference to the block diagram shown in FIG. 2. In this connection, the abnormality processing system 1 may include other elements than the configuration shown in FIG. 2 or omit part of the configuration shown in FIG. 2.

The management computer 2 includes a communication unit 5, a display unit 6, an input unit 7, an abnormality information database 8, and a processing circuit 9.

The processing circuit 9 includes an abnormality information identification unit 10, a processing method identification unit 11, a processing time identification unit 12, and a contact identification unit 13. The processing method identification unit 11 includes a times calculation unit 14, a processing date determination unit 15, and an order identification unit 16. These units can be realized using programs, which are stored in memory or a hard disk drive (HDD) and executed by a CPU (Central Processing Unit).

For example, the processing circuit 9 is circuitry including a CPU (Central Processing Unit), a GPU (Graphics Processing Unit), or a specific-purpose or general-purpose processor. The processor can realize various types of functions by executing various types of programs. In addition, the processing circuit 9 may be configured with hardware such as a FPGA (Field Programmable Gate Array) or an ASIC (Application Specific Integrated Circuit). Various types of functions can be realized by these hardware components. Alternatively, the processing circuit 9 can realize various types of functions by a combination of hardware processing and software processing using the processor and programs. Moreover, the abnormality processing method of the present embodiment can be realized by executing various types of programs on the processing circuit 9.

Each configuration of the abnormality processing system 1 is not necessarily provided with one management computer 2. For example, one abnormality processing system 1 may be realized by a plurality of management computers 2 mutually connected through the network 3. For example, the abnormality processing system 1 may be implemented on each individual management computer 2.

The communication unit 5 is configured to make communication with the other computers 4 through the network 3. For example, the network 3 corresponds to communication lines in a LAN (Local Area Network). In this connection, the network 3 may correspond to the Internet, a WAN (Wide Area Network), a mobile communication network, or the like.

The display unit 6 is configured to output the predetermined information. The management computer 2 includes the display unit 6 serving as a device for displaying images, such as a display for outputting information.

The input unit 7 is configured to input the predetermined information in response to an operation of the user who uses the management computer 2. The input unit 7 may include various input devices such as a mouse, a keyboard, and a touch panel. That is, the predetermined information will be inputted into the management computer 2 in response to the operation of input devices.

The input unit 7 cooperates with the display unit 6 to perform an information input process. For example, the display unit 6 and the input unit 7 accept a user's input operation by way of a graphical user interface (GUI). The display unit 6 displays a predetermined browser such that the user can designate a screen image with a mouse pointer and perform an operation to select and input information.

The abnormality information database 8 refers to a collection of information data, which are stored in memory, HDDs, or resources of cloud computing and organized to achieve searching or accumulation. The abnormality information database 8 shall not necessarily be stored in a storage unit such as HDDs inside the management computer 2 and can be stored in resources of cloud computing. That is, the abnormality information database 8 can be stored at any place having accessibility with the management computer 2.

The abnormality information database 8 registers a plurality of handling items representing various types of processing methods for the user to handle any abnormality of a particle beam treatment apparatus. In this connection, it is possible to register handling items with the abnormality information database 8 in advance or to arbitrarily register them by the user.

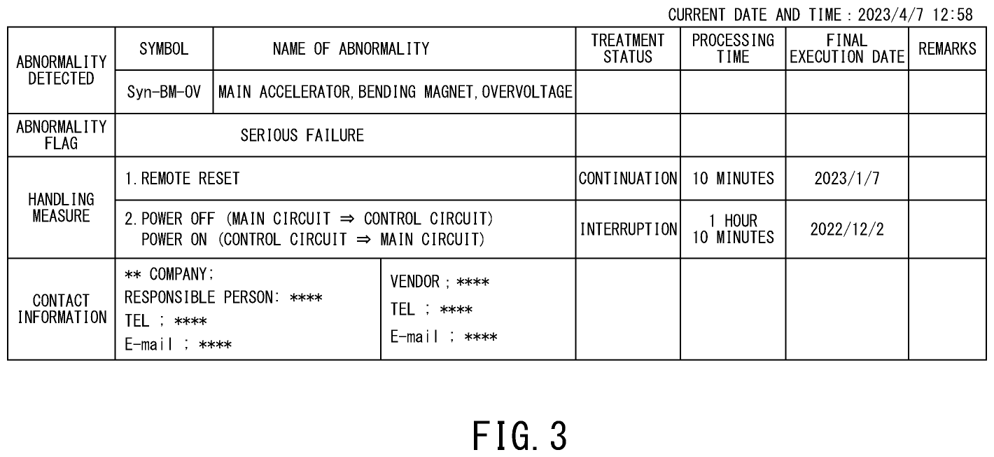

As shown in FIG. 3, the display unit 6 of the management computer 2 is configured to display various types of handling items when an abnormality occurs in a particle beam treatment apparatus. The information for generating a screen image is registered with the abnormality information database 8. As one handling item, for example, a symbol for identifying abnormality is registered with the abnormality information database 8. The symbol means identification information (or abnormality information) that can identify individual pieces of abnormality. Using the symbol as a primary key to a management table, various types of information are registered with the abnormality information database 8. For registering handling items responsive to abnormality newly occurred, the management computer 2 is configured to automatically generate a symbol.

The abnormality information database 8 is configured to register various pieces of information (or handling items) such as an abnormality flag, a handling measure, contact information, a name of abnormality, treatment status, processing time, final execution date, and remarks in association with each symbol. In this connection, it is possible to register these pieces of information with the abnormality information database 8 in advance or to register or update them in real time.

The abnormality information database 8 may register the recovery history indicating whether the user has succeeded in recovery or not by executing a predetermined processing method in the past. For example, the abnormality information database 8 may register the date (or the number of times) of executing processing methods by the user in the past, the number of times (or the effective number of times) of making successful recovery, and the number of times of making failures in recovery (or the number of times of failures).

When the user has executed a predetermined processing method in the past, the abnormality information database 8 may register other pieces of information indicating whether the processing method was effective and how long the processing method took time in the past.

For example, the abnormality flag means the information indicating the type of abnormality, i.e., the information about the classification between serious abnormality and slight abnormality. The handling measure means the information indicating an operation that the user should perform. The contact information means the information about any person who the user needs to contact with, such as a person of the company responsive to maintenance of a particle beam treatment apparatus or a vendor serving as a manufacturer of a particle beam treatment apparatus. The name of abnormality means the title corresponding to a specific type of abnormality. The treatment status means the information indicating whether to continue or interrupt treatment for a patient when the user executes the handling measure. The treatment status is not necessarily limited to the information registered in advance but also the information arbitrarily updated. The processing time means the information about the time required for the user to execute a handling measure (e.g., the time required for recovery). The final execution date means the information about a date when the user has taken a predetermined handling measure most recently. The remarks mean arbitrary information that was inputted by the user or any person concerned such as the vendor. In this connection, the term “continue” means restarting treatment on the same day without bringing a patient out of a treatment couch. The term “interrupt” means bringing a patient out of a treatment couch and restarting treatment on a later date or several hours later.

Conventional technology has a limitation in the number of characters displayable on a screen due to the occurrence of abnormality, and therefore the status of abnormality is displayed with a specific symbol irrespective of the significance of abnormality, e.g., slight abnormality or serious failure. It is pointed out that symbols may have difficult meanings, which might be hardly understood by users. In addition, it is uncertain about what the user should confirm in first determination after the occurrence of abnormality and whether the user should handle recovery. In addition, it takes a long time to make time-series analysis of phenomena, summarization, and acquisition of logs. The present embodiment can solve the above problem.

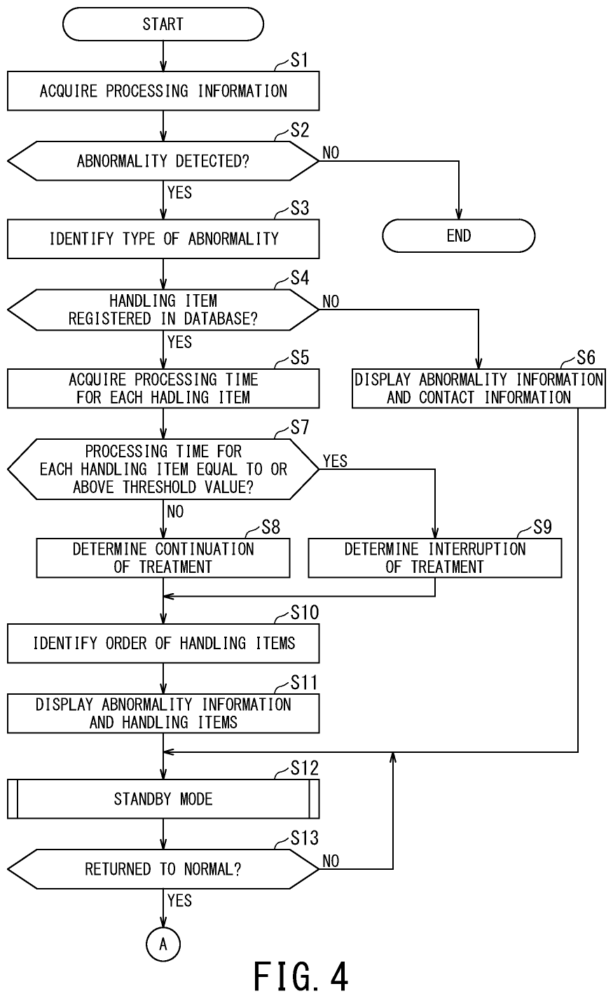

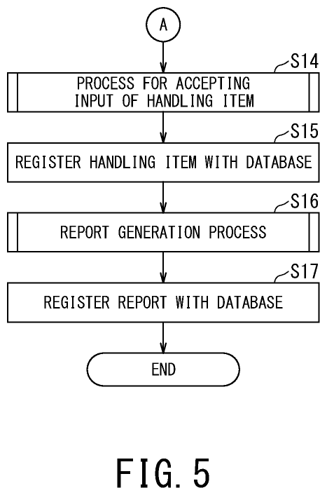

Next, an abnormality information display process executed by the management computer 2 will be described with reference to the flowcharts of FIGS. 4-5. In this connection, the following descriptions refer to the foregoing drawings. This process is a process repeated at regular intervals.

As shown in FIG. 4, in step S1, the abnormality information identification unit 10 (see FIG. 2) is configured to acquire the processing information representative of the current status of a particle beam treatment apparatus from the other computers 4 (see FIG. 1) respectively.

In the next step S2, the abnormality information identification unit 10 detects as to whether the processing information indicating any abnormality is included in the acquired processing information or not. That is, the abnormality information identification unit 10 detects any abnormality of a particle beam treatment apparatus based on the acquired processing information. When the processing information indicating any abnormality is included in the acquired processing information (i.e., when YES in step S2), the flow proceeds to step S3. In contrast, when the processing information indicating any abnormality is not included in the acquired processing information (i.e., when NO in step S2), the abnormality information display process shall end.

In the next step S3, the abnormality information identification unit 10 identifies the abnormality information registered with the abnormality information database 8 in association with the detected abnormality. For example, upon detecting abnormality, the abnormality information identification unit 10 identifies the abnormality information from the acquired processing information. In addition, the abnormality information identification unit 10 identifies the type of abnormality from the abnormality information.

In the next step S4, the processing method identification unit 11 (see FIG. 2) identifies a processing method registered with the abnormality information database 8 in association with the identified abnormality information. The processing method identification unit 11 identifies the occurrence of the already known abnormality or the new abnormality. For example, the processing method identification unit 11 determines as to the existence or nonexistence of any handling item registered with the abnormality information database 8 in association with the identified type of abnormality. Upon determining the existence of any handling item associated with the identified type of abnormality (i.e., when YES in step S4), the flow proceeds to step S5. In contrast, upon determining the nonexistence of any handling item associated with the identified type of abnormality (i.e., when NO in step S4), the flow proceeds to step S6.

In step S5, the processing time identification unit 12 (see FIG. 2) acquires the processing time of a handling item associated with the identified type of abnormality. Upon detecting a single handling item, the processing time identification unit 12 acquires the processing time registered with the abnormality information database 8 in association with a single handling item. Upon detecting multiple processing items, the processing time identification unit 12 acquires the processing times registered with the abnormality information database 8 in association with multiple handling items respectively.

In addition, the processing time identification unit 12 calculates the processing time representative of any one of average time, maximum time, and minimum time required in the processing method. The processing time may be calculated and registered with the abnormality information database 8 in advance.

The processing method identification unit 11 may further acquire a symbol (or abnormality information), an abnormality flag, a handling measure, contact information, the name of abnormality, a treatment status, a final execution date, and other information such as remarks (or handling items) together with the processing time, and then the flow proceeds to step S7.

In step S6, the processing method identification unit 11 generates a symbol (or abnormality information) indicating new abnormality and registers the symbol with the abnormality information database 8. Then, the processing method identification unit 11 displays the symbol (or abnormality information). The contact identification unit 13 (see FIG. 2) identifies the contact information of a vendor or the like registered with the abnormality information database 8, displays the contact information on the display unit 6 (see FIG. 2), and then the flow proceeds to step S12 described later.

In step S7, the processing time identification unit 12 compares the processing time of a handling item with a threshold value set in advance. For example, the processing time identification unit 12 determines as to whether the processing time is equal to or above the threshold value or not. When the processing time is below the threshold value (i.e., when NO in step S7), the flow proceeds to step S8. In contrast, when the processing time is equal to or above the threshold value (i.e., when YES in step S7), the flow proceeds to step S9. Upon detecting multiple handling items, the processing time identification unit 12 determines multiple processing times for multiple processing items (via multiple times of determination).

In step S8, the processing time identification unit 12 determines “continue” as the treatment status associated with a handling item having the processing time below the threshold value, and then the flow proceeds to step S10.

In step S9, the processing time identification unit 12 determines “interrupt” as the treatment status associated with a handling item having the processing time equal to or above the threshold value, and then the flow proceeds to step S10.

Since the management computer 2 is configured to determine continuation or interruption of treatment for a patient upon comparing the processing time with the threshold value set in advance, it is possible to objectively determine continuation or interruption of treatment for a patient by use of the threshold set in advance.

Since the management computer 2 determines continuation or interruption of treatment for a patient based on the processing time required to handle abnormality, it is possible to objectively determine continuation or interruption of treatment for a patient based on the processing time required to handle abnormality. The above determination can be arbitrarily made by the user according to the condition of a patient.

Upon detecting multiple handling items, it is necessary to determine multiple processing times in an order of higher priority. At first, for example, it is determined whether the processing time corresponding to the processing method having the first place of priority is equal to or above the threshold value or not. Next, it is determined whether the total of processing times corresponding to processing methods having the first place of priority and the second place of priority is equal to or above the threshold value or not. Next, it is determined whether the total of processing times corresponding to processing methods having the first place of priority to the third place of priority is equal to or above the threshold value or not.

In step S10, the order identification unit 16 (see FIG. 2) identifies an order of handling items, that is, an order of processing methods recommended. Upon detecting multiple handling items, the order identification unit 16 identifies an order of handling items, which the management computer 2 preferentially displays on the display unit 6, according to the condition set in advance. By doing so, the user can proceed with the recovery work according to a priority order of handling items.

For example, the order identification unit 16 identifies an order to preferentially display a handling item associated with the continuation of treatment on the display unit 6. By doing so, it is possible to prevent the treatment for a patient from being interrupted as much as possible.

The order identification unit 16 identifies an order such that a handling item with the highest probability of success in recovery will be preferentially displayed on the display unit 6. The times calculation unit 14 calculates the effective number of times (or the rate) of carrying out handling items against abnormality among handling items registered with the abnormality information database 8. For example, multiple handling items can be registered with the abnormality information database 8 in association with a predetermined type of abnormality. In this case, the order identification unit 16 orders multiple handling items in a descending order of the higher probability in success of recovery calculated by the times calculation unit 14 or in a descending order of the larger effective number of times calculated by the times calculation unit 14. This makes it possible to preferentially display a handling item with the highest probability of success in recovery on the display unit 6, and therefore the user can proceed with recovery work according to the most appropriate processing method.

For example, the order identification unit 16 orders handling items such that a higher probability of success in recovery (or a larger effective number of times) would be applied to a handling item, which may achieve success in recovery four times among five-times attempts, rather than a handling item, which may achieve success in recovery five times among ten-times attempts.

The order identification unit 16 identifies an order to preferentially display a handling item, which is executed on the most recent date, on the display unit 6. For example, the processing date determination unit 15 (see FIG. 2) determines an execution date for the user to execute the processing method in the past with respect to each handling item. Then, the order identification unit 16 orders multiple handling items in a descending order of execution dates counted from the newest execution date. This may verify that a handling item executed on the most recent date close to the present date has the highest probability of success in recovery, and therefore the user can proceed with recovery work according to the processing method.

The order identification unit 16 may order multiple handling items based on the processing times calculated by the processing time identification unit 12. For example, upon identifying multiple handling items, the processing time identification unit 12 may calculate the processing time required to execute the second handling item as the total of the time required to execute the first handling item and the time required to execute the second handling item. Then, the order identification unit 16 orders multiple handling items in a descending order of processing times counted from the shortest processing time. This may allow the user to grasp the total time required to execute the second and subsequent handling items, thus sequentially executing the processing methods starting with the processing method having the shortest processing time. This process can be performed before the determination of step S7.

In case of multiple types of orders, the order identification unit 16 prioritizes a handling item having the shortest processing time rather than a handling item executed on the most recent date close to the present date. In addition, the order identification unit 16 prioritizes a handling item having the highest probability of success in recovery rather than a handling item having the shortest processing time. Moreover, the order identification unit 16 prioritizes a handling item associated with the continuation of treatment rather than a handling item having the highest probability of success in recovery. In this connection, the above orders are illustrative, and therefore it is possible to adopt other manners as to how to determine orders.

In the next step S11, the processing method identification unit 11 displays a symbol indicating abnormality (or abnormality information) and its corresponding handling item on the display unit 6. Herein, handling measures are displayed in an order determined by the order identification unit 16. In addition, the contact identification unit 13 (see FIG. 2) identifies the contact information of a vendor or the like registered with the abnormality information database 8, thus displaying the contact information on the display unit 6.

The processing method identification unit 11 displays the processing time of the processing method for each handling item on the display unit 6. For example, the display unit 6 may display at least one of average time, maximum time, and minimum time required in the processing method, or the time set for the processing method in advance.

As shown in FIG. 3, the management computer 2 displays various types of handling items on the screen. For example, the management computer 2 displays handling items including the information helping the user to determine whether to “continue” or “interrupt” the treatment for a patient.

The information helping the user to determine whether to continue or interrupt the treatment for a patient may refer to not only a display of “continue” or “interrupt” but also a display of “processing time”. Without a display of “continue” or “interrupt”, the user can determine whether to continue or interrupt the treatment for a patient by grasping “processing time”. When displaying “processing time”, it is possible to change the color thereof or to highlight the message thereof, thus allowing the user to recognize whether “processing time” is equal to or above a threshold value or not by way of coloring or highlighting.

Since the management computer 2 identifies a handling item, which is registered with the abnormality information database 8 in association with the identified type of abnormality, to thereby display the identified handling item, it is possible to provide the user with an appropriate processing method according to the type of abnormality.

Returning to FIG. 4, in the next step S12, the abnormality information identification unit 10 makes a transition to a standby mode of waiting for a predetermined time while the user is executing a processing method. Herein, the abnormality information identification unit 10 acquires the processing information indicating the current status of a particle beam treatment apparatus from each of other computers 4 (see FIG. 1).

In the next step S13, the abnormality information identification unit 10 determines, based on the acquired processing information, whether the particle beam treatment apparatus has returned to normal or not. In the case of the particle beam treatment apparatus returned to normal (i.e., when YES in step S13), the flow proceeds to step S14. In contrast, if the particle beam treatment apparatus has not returned to normal (i.e., when NO in step S13), the flow returns to step S12 representative of the standby mode.

As shown in FIG. 5, in step S14, the abnormality information identification unit 10 receives an input of a handling item from the user. For example, the user may select a handling item already displayed on the screen (see FIG. 3), or the user may newly input a handling item.

Without displaying handling items on the screen, it is possible for the user to input the date representative of the occurrence of abnormality, the content of the processing method actually performed, the time required for processing, and the information indicating whether the processing was effective or not via the input unit 7 (see FIG. 2). In addition, the user may input the contact information and the information about logs confirmed before the processing via the input unit 7. The contact information may include the information used to make an inquiry with another vendor or another person responsible other than any person listed in the contact information displayed on the display unit 6 (see FIG. 2). The new handling item may include all processing methods that the user has performed until a particle beam treatment apparatus returns to normal. For example, the new handling item may include not only the processing method achieving success in recovery but also the processing method failed in recovery.

In the next step S15, the abnormality information identification unit 10 registers a handling item selected by the user or a new handling item inputted by the user with the abnormality information database 8. By doing so, when the user recovers a particle beam treatment apparatus according to another method than the handling item displayed on the management computer 2, it is possible to register another method as the new handling item with the abnormality information database 8.

In the next step S16, the abnormality information identification unit 10 executes a process to generate and output a report. Herein, the abnormality information identification unit 10 generates a report based on a handling item indicated by the user among multiple handling items displayed on the display unit 6. By doing so, it is possible to reduce time and labor of the user to produce the report. In addition, the abnormality information identification unit 10 displays the report on the display unit 6.

In the next step S17, the abnormality information identification unit 10 registers the generated report and the content thereof with the abnormality information database 8.

Then, the management computer 2 exits the abnormality information display process. The above steps correspond to at least part of steps included in the abnormality information display process, and therefore other steps can be included in the abnormality information display process.

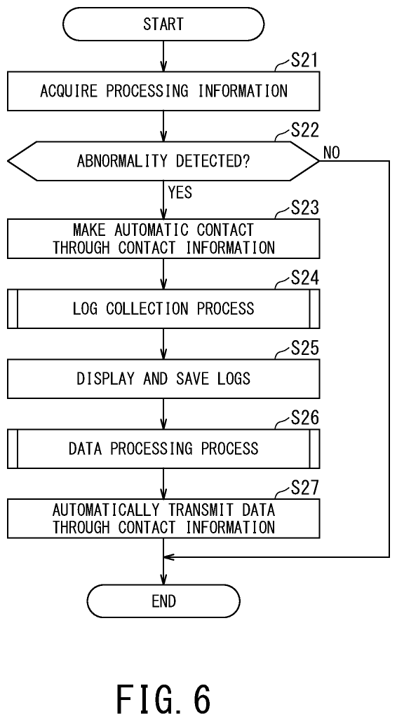

Next, a log collection process executed by the management computer 2 will be described with reference to the flowchart of FIG. 6. In this connection, the log collection process may refer to the foregoing drawings. This process is a process repeated at regular intervals.

As shown in FIG. 6, in step S21, the abnormality information identification unit 10 (see FIG. 2) acquires the processing information indicating the current status of a particle beam treatment apparatus from the other computers 4 (see FIG. 1).

In the next step S22, the abnormality information identification unit 10 determines whether any abnormality has been detected in the particle beam treatment apparatus or not, based on the acquired processing information. Upon detecting abnormality (i.e., when YES in step S22), the flow proceeds to step S23. In contrast, if abnormality has not been detected (i.e., when NO in step S22), the management computer 2 exits the log collection process.

In step S23, the contact identification unit 13 (see FIG. 2) identifies the contact information such as a vendor registered with the abnormality information database 8 and notifies the occurrence of abnormality to the personnel of the contact information. For example, the contact identification unit 13 automatically transmits an email stating the occurrence of abnormality (e.g., a first notification) to an email address of the contact information identified. In this connection, the first notification is not necessarily limited to communication via an email, but it is possible to adopt any other means. For example, it is possible to use another predetermined contact set in advance rather than the contact identified by the contact identification unit 13.

In the next step S24, the abnormality information identification unit 10 collects logs. For example, logs may correspond to logs of any equipment or any system relating to abnormality identified from abnormality information. The log collection process will be continuously executed during a period counted from the timing of the occurrence of abnormality to the timing of recovery. In this connection, the collected logs include not only the logs automatically collected by the management computer 2 but also other logs manually collected and inputted into the management computer 2 by the user.

The occurrence of abnormality in a particle beam treatment apparatus may result in stoppage of another apparatus (not shown) via interlocking or in occurrence of another abnormality. When an interlocking is activated by another related apparatus simultaneously with the occurrence of abnormality in a particle beam treatment apparatus, the abnormality information identification unit 10 shall collect logs about another related apparatus.

In the next step S25, the abnormality information identification unit 10 displays the collected logs on the display unit 6 (see FIG. 2) and registers (or saves) the collected logs with the abnormality information database 8. In this connection, it is possible to store the collected logs on a predetermined storage unit (not shown) other than the abnormality information database 8.

The abnormality information identification unit 10 registers the foregoing information relating to another apparatus with the abnormality information database 8 in association with the identified type of abnormality. By doing so, it is possible to record an abnormality of another apparatus that may occur incidentally to the abnormality of a particle beam treatment apparatus. Then, the abnormality information identification unit 10 registers the abnormality of another apparatus that occurs incidentally with the abnormality information database 8 in association with the abnormality information.

In this connection, it is possible to adopt a different display manner than the normal display manner of abnormality with respect to plenty of abnormalities that may occur in relation to a single abnormality of a particle beam treatment apparatus. When displaying the related information on the display unit 6 (FIG. 2), for example, the abnormality information identification unit 10 may change the color thereof or highlight the message thereof.

In the next step S26, the abnormality information identification unit 10 processes data including the collected logs. For example, the abnormality information identification unit 10 may execute a process of itemizing and encrypting necessary ones of the collected logs.

In the next step S27, the contact identification unit 13 identifies the contact information such as a vendor registered with the abnormality information database 8 and automatically transmits the processed data including the collected logs (e.g., a second notification) to the personnel of the contact information.

Then, the management computer 2 exits the log collection process. The above steps correspond to at least part of the steps included in the log collection process, and therefore the log collection process may include other steps.

In this connection, the foregoing flowcharts exemplarily show a series of steps to be executed serially, however, which may not necessarily fix the fore-and-aft relationship of steps; hence, part of steps can be changed in the fore-and-aft relationship thereof. In addition, it is possible to execute part of steps in parallel with another part of steps.

The abnormality processing system 1 described above includes a control device, a storage device, an output device, an input device, and a communication interface. The control device refers to a highly integrated processor, such as CPU (Central Processing Unit), GPU (Graphics Processing Unit), FPGA (Field Programmable Gate Array), and other specific chips. The storage device refers to ROM (Read Only Memory), RAM (Random Access Memory), HDD (Hard Disk Drive), and SSD (Solid State Drive). The output device refers to a display panel, a head-mounted display, a projector, and a printer. The input device refers to a mouse, a keyboard, and a touch panel. The abnormality processing system 1 can be realized using a hardware configuration utilizing a common-use computer.

It is possible to provide the abnormality processing system 1 with programs using ROM configured to store programs in advance. Additionally, or alternatively, it is possible to provide programs in an installable format of files, or an executable format of files stored in computer-readable non-transitory storage media. The storage media refer to CD-ROM, CD-R, DVD, a memory card, and a flexible disk (FD).

In addition, it is possible to store the programs executed by the abnormality processing system 1 on a computer connected to the network 3 such as the Internet and to provide the programs downloaded thereto through networks. Moreover, the abnormality processing system 1 can be configured with separate modules for demonstrating functions of constituent elements individually, which are mutually connected through the network 3 or other specific lines and thereby combined in functionality.

The foregoing embodiments are designed to display handling items including information enabling each user to determine whether to continue or interrupt a treatment for a patient, and therefore, it is possible to display handling items enabling each user to immediately determine whether to continue treatment or not when any abnormality occurs in a particle beam treatment apparatus.

Although the present invention has been described above with reference to the foregoing embodiments, the foregoing embodiments are illustrative and do not intend to limit the scope of the invention. The foregoing embodiments can be practiced in other various forms, and therefore it is possible to realize various omissions, replacements, changes, and combinations within the range not departing from the spirit of the invention. The embodiments and modifications will be included in the scope and the spirit of the invention and included in the invention as defined in claims and equivalents thereof. In this connection, any term described in a singular expression will not necessarily be intended to be a singular form, since a singular form can be redefined as a multiple form.

1. An abnormality processing system comprising one or more management computers connected to a plurality of computers configured to process information relating to a particle beam treatment apparatus through a network, wherein the management computer is configured to:
acquire processing information indicating a current status of the particle beam treatment apparatus from the plurality of computers,
detect, when at least one abnormality occurs in the particle beam treatment apparatus, the abnormality from the acquired processing information, and
display, upon detecting the abnormality, at least one handling item indicating a processing method for a user to process the abnormality, which includes information enabling the user to determine whether to continue or interrupt a treatment for a patient.

2. The abnormality processing system according to claim 1, comprising a database for registering a plurality of handling items indicating various processing methods for the user to process the abnormality, wherein the management computer is configured to:
identify, upon detecting the abnormality, a type of the abnormality from the acquired processing information,
identify the handling item registered with the database in association with the identified type of the abnormality, and
display the identified handling item.

3. The abnormality processing system according to claim 1, wherein upon detecting the abnormality, the management computer is configured to:
determine whether to continue or interrupt the treatment for the patient based on processing time required to process the abnormality, and
display a determination result as the handling item.

4. The abnormality processing system according to claim 3, wherein the management computer is configured to determine whether to continue or interrupt the treatment for the patient by comparing the processing time with a threshold value set in advance.

5. The abnormality processing system according to claim 4, wherein upon detecting the abnormality to identify a plurality of handling items based on the acquired processing information, the management computer is configured to calculate the processing time required to execute a second handling item as a total of a time required to execute a first handling item and a time required to execute a second handling item.

6. The abnormality processing system according to claim 1, wherein upon detecting the abnormality, the management computer is configured to display a processing time required to process the abnormality.

7. The abnormality processing system according to claim 1, wherein upon detecting the abnormality to identify a plurality of handling items based on the acquired processing information, the management computer is configured to identify an order of handling items to be prioritized and displayed based on a condition set in advance.

8. The abnormality processing system according to claim 1, wherein upon detecting the abnormality to identify a plurality of handling items based on the acquired processing information, the management computer is configured to preferentially display the handling item associated with continuation of the treatment.

9. The abnormality processing system according to claim 1, wherein upon detecting the abnormality to identify a plurality of handling items based on the acquired processing information, the management computer is configured to preferentially display the handling item having a highest probability of success in recovery.

10. The abnormality processing system according to claim 1, wherein upon detecting the abnormality to identify a plurality of handling items based on the acquired processing information, the management computer is configured to preferentially display the handling item executed on a most recent date close to a present date.

11. The abnormality processing system according to claim 2, wherein the management computer is configured to register a new handling item inputted by the user with the database.

12. The abnormality processing system according to claim 2, wherein when interlocking is activated in another apparatus simultaneously with occurrence of the abnormality in the particle beam treatment apparatus, the management computer is configured to register information about another apparatus with the database in association with the identified type of the abnormality.

13. The abnormality processing system according to claim 1, wherein the management computer is configured to generate a report based on the handling item indicated by the user among a plurality of handling items displayed on a screen.

14. An abnormality processing method executed by one or more management computers connected to a plurality of computers configured to process information related to a particle beam treatment apparatus through a network, wherein the management computer is configured to:
acquire processing information indicating a current status of the particle beam treatment apparatus from the plurality of computers;
detect, when at least one abnormality occurs in the particle beam treatment apparatus, the abnormality from the acquired processing information; and
display, upon detecting the abnormality, at least one handling item indicating a processing method for a user to process the abnormality, which includes information enabling the user to determine whether to continue or interrupt a treatment for a patient.

15. The abnormality processing system according to claim 2, wherein upon detecting the abnormality, the management computer is configured to:
determine whether to continue or interrupt the treatment for the patient based on processing time required to process the abnormality, and
display a determination result as the handling item.

16. The abnormality processing system according to claim 15, wherein the management computer is configured to determine whether to continue or interrupt the treatment for the patient by comparing the processing time with a threshold value set in advance.

17. The abnormality processing system according to claim 16, wherein upon detecting the abnormality to identify a plurality of handling items based on the acquired processing information, the management computer is configured to calculate the processing time required to execute a second handling item as a total of a time required to execute a first handling item and a time required to execute a second handling item.

18. The abnormality processing system according to claim 2, wherein upon detecting the abnormality, the management computer is configured to display a processing time required to process the abnormality.

19. The abnormality processing system according to claim 2, wherein upon detecting the abnormality to identify a plurality of handling items based on the acquired processing information, the management computer is configured to identify an order of handling items to be prioritized and displayed based on a condition set in advance.

20. The abnormality processing system according to claim 2, wherein upon detecting the abnormality to identify a plurality of handling items based on the acquired processing information, the management computer is configured to preferentially display the handling item associated with continuation of the treatment.

21. The abnormality processing system according to claim 2, wherein upon detecting the abnormality to identify a plurality of handling items based on the acquired processing information, the management computer is configured to preferentially display the handling item having a highest probability of success in recovery.

22. The abnormality processing system according to claim 2, wherein upon detecting the abnormality to identify a plurality of handling items based on the acquired processing information, the management computer is configured to preferentially display the handling item executed on a most recent date close to a present date.

23. The abnormality processing system according to claim 2, wherein the management computer is configured to generate a report based on the handling item indicated by the user among a plurality of handling items displayed on a screen.
